(19) United States
(12) Patent Application Publication
(10) Pub. No.: US 2022/0296676 A1
(43) Pub. Date: Sep. 22, 2022

(54) TREATMENT OR PREVENTION OF CORONAVIRIDAE INFECTION

(71) Applicant: WATERSTONE PHARMACEUTICALS(WUHAN) CO., LTD., Wuhan, Hubei (CN)

(72) Inventors: Jian CUI, Wuhan, Hubei (CN); Minglong HU, Wuhan, Hubei (CN); Yao YU, Wuhan, Hubei (CN); Along ZHAO, Wuhan, Hubei (CN); Faming ZHANG, Wuhan, Hubei (CN)

(21) Appl. No.: 17/429,706

(22) PCT Filed: Jun. 4, 2020
(86) PCT No.: PCT/CN2020/094431

§ 371 Date: Aug. 10, 2021

(51) Int. Cl. A61K 38/13; A61P 31/14

(52) U.S. Cl. CPC A61K 38/13; A61P 31/14

(57) ABSTRACT

A method of treating or preventing a Coronaviridae infection in a subject comprising administrating a therapeutically effective amount of a compound of Formula I or a stereoisomer, a tautomer, an N-oxide, a solvate, a metabolite, a pharmaceutically acceptable salt or a prodrug thereof, and the Coronaviridae comprises at least one selected from 2019-nCov virus, HCov 229E virus, SARS virus, MERS virus,

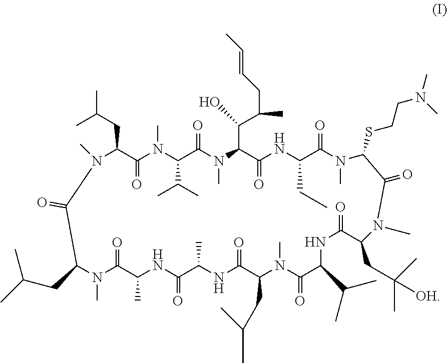 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### FIELD OF THE INVENTION

The present invention relates to the field of medicinal technology, in particular, to the treatment or prevention of Coronaviridae infection in a subject.

### BACKGROUND OF THE INVENTION

The World Health Organization has recently declared the severe acute respiratory syndrome coronavirus 2 (SARS-Cov-2) a public health emergency of international concern. As of Jun. 1, 2020, there are more than 6,000,000 confirmed cases and more than 370,000 death cases all around the world.

Coronaviridae (also may be known as “Coronaviruses”) is a family of enveloped, positive-sense, single-stranded RNA viruses. Coronaviruses may cause diseases in mammals and birds. In humans, the viruses cause respiratory infections, including the common cold, which are typically mild, though rarer forms such as SARS (including the one causing COVID-19) and MERS can be lethal.

Therefore there is an increasing demand for the research and development of more effective method for treating or preventing a Coronaviridae infection.

### SUMMARY OF THE INVENTION

The following is only an overview of some aspects of the present invention, but is not limited thereto. All references of this specification are incorporated herein by reference in their entirety. When the disclosure of this specification is different with citations, the disclosure of this specification shall prevail.

In the research and development process, the inventors surprisingly found that WS-635 (the compound of formula (I)) showed significantly high efficiency and activity for treating or preventing a Coronaviridae infection in several experimental tests.

In one aspect of present disclosure, there is provided a method of treating or preventing a Coronaviridae infection in a subject comprising administrating a therapeutically effective amount of a compound of Formula I or a stereoisomer, a tautomer, an N-oxide, a solvate, a metabolite, a pharmaceutically acceptable salt or a prodrug thereof to the subject in need thereof,

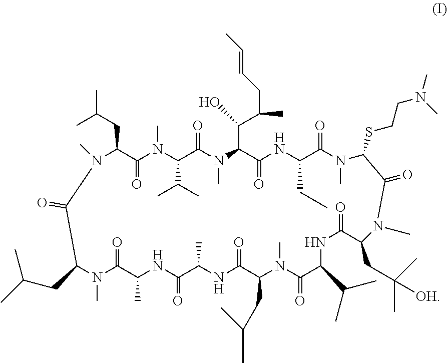 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

and the Coronaviridae comprises at least one selected from 2019-nCov virus, HCov 229E virus, SARS virus, MERS virus.

In another aspect of present disclosure, there is provided a pharmaceutical composition for treating or preventing Coronaviridae virus infection comprising a compound of Formula I or a stereoisomer, a tautomer, an N-oxide, a solvate, a metabolite, a pharmaceutically acceptable salt or a prodrug thereof,

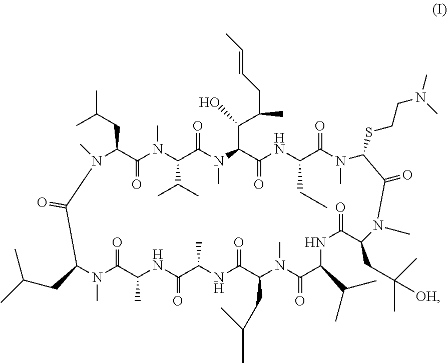 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

and the Coronaviridae comprises at least one selected from 2019-nCov virus, HCov 229E virus, SARS virus, MERS virus.

In another aspect of present disclosure, there is provided compound of Formula I or a stereoisomer, a tautomer, an N-oxide, a solvate, a metabolite, a pharmaceutically acceptable salt or a prodrug thereof, for use in treating or preventing Coronaviridae infection in a subject,

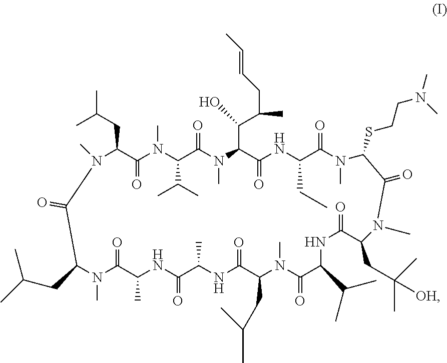 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

the Coronaviridae comprises at least one selected from 2019-nCov virus, HCov 229E virus, SARS virus, MERS virus.

In another aspect of present disclosure, there is provided a use of a compound of Formula I or a stereoisomer, a tautomer, an N-oxide, a solvate, a metabolite, a pharmaceutically acceptable salt or a prodrug thereof in the manufacture of a medicament for treating or preventing Coronaviridae infection.

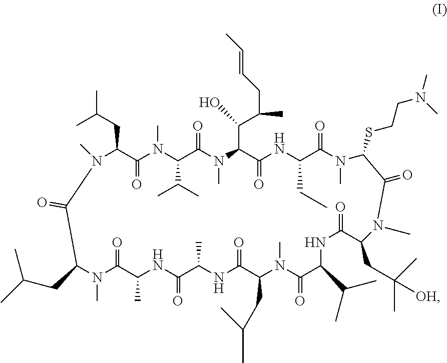 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

the Coronaviridae comprises at least one selected from 2019-nCov virus, HCov 229E virus, SARS virus, MERS virus.

The foregoing merely summarizes certain aspects disclosed herein and is not intended to be limiting in nature. These aspects and other aspects and additional embodiments, features, and advantages of the invention will be apparent from the following detailed description and through practice of the invention.

### BRIEF DESCRIPTION OF THE DRAWINGS

These and other aspects and advantages of embodiments of the present disclosure will become apparent and more readily appreciated from the following descriptions made with reference the accompanying schemes and drawings, in which:

FIG. 1 shows the inhibitory effect of WS-635 on the SARS-CoV-2 coronavirus at the cellular level;

FIG. 2 shows WS-635 anti SARS-CoV-2 activity results; and

FIG. 3 shows WS-635 anti human coronavirus (HCoV) 229E activity results.

### MOBODIMENT

### Definition

Unless otherwise indicated, otherwise the following terms and phrases as used herein will have the following meanings:

“Esters” refers to any ester of the compound, wherein the molecule of any-COOH functional group is —C(O) OR function with a substituent, or wherein any of the molecule-OH functional group is —OC(O) R functional groups, wherein R may be formed a stable ester moiety of the ester moiety is any carbon-containing groups, including but not limited to alkyl, alkenyl, alkynyl, cycloalkyl, cycloalkylalkyl, aryl, arylalkyl, heterocyclyl, heterocycloalkyl and substituted derivatives thereof.

“Pharmaceutically acceptable” refers to a suitable for use in a pharmaceutical formulation, generally regarded as safe, a regulatory agency of the country or a state government official authorized to be used by, or column in the Pharmacopeia or other generally recognized pharmacopeia for use in animals and particularly for use in humans.

“Pharmaceutically acceptable carrier” refers to a pharmaceutically acceptable diluent, adjuvant, excipient or carrier or other component, and is administered in combination with a compound of the present invention.

“Pharmaceutically acceptable salt” refers to the desired pharmacologically active salt thereof can be enhanced. Pharmaceutically acceptable salts thereof with inorganic or organic acids include the acid addition salts, metal salts and amine salts. Acid addition salts may be formed with inorganic acids and organic acids.

“Prodrug” refers to a biological system at the time of administration, as a spontaneous chemical reaction, an enzyme-catalyzed chemical reactions, photolysis and/or metabolic result of chemical processes, can generate any compound of which the active ingredient drug substance. Thus, the prodrug is a therapeutically active compound covalently modified analogs or latent form. A non-limiting example includes a pro-drug ester, quaternary ammonium molecules, glycol molecules and the like.

“a therapeutically effective amount” means the compound was administered to an animal for the treatment of diseases, the treatment of the disease in an amount sufficient to effect.

A therapeutically effective amount of a compound “treating” means and includes any of the application:

(1) prevention may be predisposed to the disease but still does not yet experience or display the pathology or symptomatology of the disease in animals that develop the disease,

(2) inhibiting the disease is experiencing or displaying the pathology or symptomatology of the disease in an animal of the (i.e., arresting further development of the pathology and/or symptomatology), or

(3) improve the pathology or symptomatology of the disease is experiencing or displaying the disease in an animal (i.e., reversing the pathology and/or symptomatology).

### Compound

The present disclosure relates to a compound of Formula I or a stereoisomer, a tautomer, an N-oxide, a solvate, a metabolite, a pharmaceutically acceptable salt or a prodrug thereof,

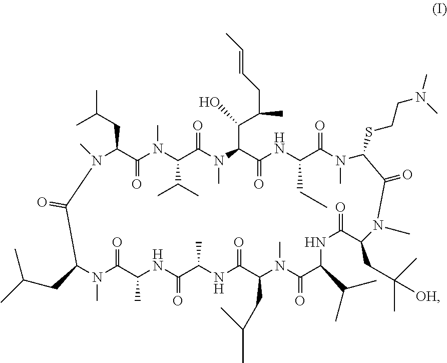 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In stereoisomer, a tautomer, an N-oxide, a solvate, a metabolite, a pharmaceutically acceptable salt or a prodrug when treating or preventing a Coronaviridae infection in a subject in need thereof And in some examples, the Coronaviridae may comprise at least one selected from 2019-nCov virus, HCov 229E virus, SARS virus, MERS virus.

In some examples of present disclosure, the compound of Formula I may be used in a form a pharmaceutically acceptable salt, and the pharmaceutically acceptable salt is at least one selected from a group comprising phosphate, hydrochloride, hydrobromide, hydroiodide, sulfate, nitrate, ethanesulfonate, toluenesulfonate and benzenesulfonate, acetate, trifluoroacetate, tartrate, maleate, succinate, citrate, benzoate, salicylate and ascorbate, adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate, galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate.

### Pharmaceutical Formulation and Use

The compound of present disclosure may be formulated with conventional carriers and excipients, carriers and excipients which will meet the selected common practice. Tablets containing excipients, glidants, fillers, binders and the like. An aqueous formulation will be made in the form of a sterile, when it is intended for administration of the administered orally, typically isotonic solution.

While the active ingredient can be administered alone, but may preferably be formulated into pharmaceutical formulation. The formulations of the present invention, whether for human or veterinary use in formulations, each having at least one active ingredient, and one or more acceptable carriers and optionally other therapeutic ingredients.

The present invention suitable for oral administration of the formulations may be presented as discrete units, each containing a predetermined quantity of active ingredient such as capsules, cachets or tablets; a powder or granules; in an aqueous or non-aqueous liquid solutions or suspensions; or oil in water emulsion or a water in oil emulsion. The active ingredients may also be a bolus, electuary or paste form.

An effective amount of an active ingredient at least depending upon the nature of the disease being treated, toxicity, the compound is used for prophylaxis (low dose) or for the active viral infection, pharmaceutical formulation and administration method, and then using a conventional dose escalation studies by the clinician to make a decision.

In some preferred embodiments of present disclosure, the present disclosure relates to the compound of Formula I. It is surprisingly found by the inventors that the compound of Formula I showed significantly high efficiency and activity for treating or preventing a Coronaviridae infection in several experimental tests.

Then the compound of Formula I or the pharmaceutically acceptable salt, ester or prodrug thereof may be administrated to the subject in a form of sol, aromatic water, aerosol, partial inhalant, powder, granule, tablets, ointment, paste, emulsion, suspension, gas dispersion, solid dispersion, microparticle, pill. And in some examples, the compound of Formula I or a pharmaceutically acceptable salt, ester or prodrug thereof may be administrated at a daily dose of lower than 1000 mg, for example 100 mg to 1000 mg, 100˜500 mg in term of the compound of Formula I.

Additionally the person skilled in the art may understand that combination therapy may also be used. The compounds of the compound of Formula I or a pharmaceutically acceptable salt, ester or prodrug thereof may be in combination with one or more active agents. Suitable active agents for use in combination a non-limiting embodiments include one or more selected from the group comprising a corticosteroid, an anti-inflammatory signal transduction modulator, a β2-adrenoreceptor agonist bronchodilator, an anticholinergic, a mucolytic agent, hypertonic saline, agent for reducing cytokine storm, other drugs for treating a covid-19 virus infection, or mixtures thereof Then in some examples, it is provided a method of treating or preventing covid-19 virus infection in a subject comprising administrating a therapeutically effective amount of a compound of Formula I or a pharmaceutically acceptable salt, ester, hydrate or prodrug thereof to the subject in need thereof

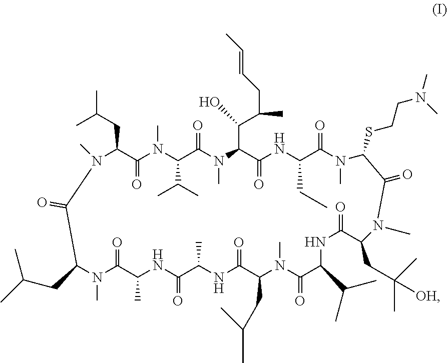 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

And in some specific examples, it is provided that the method may further comprise administering a therapeutically effective amount of at least one other therapeutic agent or composition thereof selected from the group comprising a corticosteroid, an anti-inflammatory signal transduction modulator, a β2-adrenoreceptor agonist bronchodilator, an anticholinergic, a mucolytic agent, hypertonic saline, agent for reducing cytokine storm, other drugs for treating a covid-19 virus infection, or mixtures thereof.

And in some specific examples, the compound of Formula I or the pharmaceutically acceptable salt, ester, hydrate or prodrug thereof is administrated to the subject in a form of sol, aromatic water, aerosol, partial inhalant, powder, granule, tablets, ointment, paste, emulsion, suspension, gas dispersion, solid dispersion, microparticle, pill.

And in some specific examples, compound of Formula I or a pharmaceutically acceptable salt, ester, hydrate or prodrug thereof is administrated at a daily dose of lower than 1000 mg in term of the compound of Formula I.

And in some specific examples, the compound of Formula I or a pharmaceutically acceptable salt, ester, hydrate or prodrug thereof is administrated at a daily dose of 100 mg to 1000 mg in term of the compound of Formula I.

And in some specific examples, the compound of Formula I or a pharmaceutically acceptable salt, ester, hydrate or prodrug thereof is administrated at a daily dose of 100 mg to 500 mg in term of the compound of Formula I.

In some other examples, it is provided a pharmaceutical composition to treat or prevent covid-19 virus infection comprising a compound of Formula I or a pharmaceutically acceptable salt, ester, hydrate or prodrug thereof to the subject in need thereof

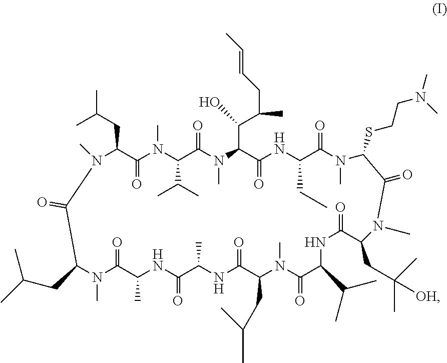 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

And in some specific examples, the composition further comprises at least one other therapeutic agent or composition thereof selected from the group comprising a corticosteroid, an anti-inflammatory signal transduction modulator, a β2-adrenoreceptor agonist bronchodilator, an anticholinergic, a mucolytic agent, hypertonic saline, agent for reducing cytokine storm, other drugs for treating a covid-19 virus infection, or mixtures thereof.

And in some specific examples, the pharmaceutically acceptable salt is at least one selected from a group comprising phosphate, hydrochloride, hydrobromide, hydroiodide, sulfate, nitrate, ethanesulfonate, toluenesulfonate and benzenesulfonate, acetate, trifluoroacetate, tartrate, maleate, succinate, citrate, benzoate, salicylate and ascorbate, adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate, galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate.

And in some specific examples, the compound of Formula I or the pharmaceutically acceptable salt, ester, hydrate or prodrug thereof is in a form of sol, aromatic water, aerosol, partial inhalant, powder, granule, tablets, ointment, paste, emulsion, suspension, gas dispersion, solid dispersion, microparticle, pill.

And in some specific examples, the composition comprises the compound of Formula I or the pharmaceutically acceptable salt, ester, hydrate or prodrug thereof at a dose of lower than 1000 mg, lower than 800 mg, lower than 600 mg, lower than 500 mg, or lower than 200 mg.

Then it is provided in the present disclosure compound of Formula I or a pharmaceutically acceptable salt, ester, hydrate or prodrug thereof, for use in treating or preventing covid-19 virus infection in a subject,

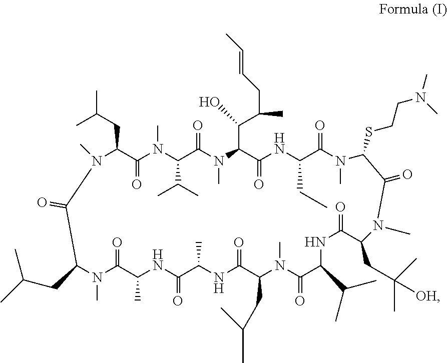 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

And in some specific examples, the pharmaceutically acceptable salt is at least one selected from a group comprising phosphate, hydrochloride, hydrobromide, hydroiodide, sulfate, nitrate, ethanesulfonate, toluenesulfonate and benzenesulfonate, acetate, trifluoroacetate, tartrate, maleate, succinate, citrate, benzoate, salicylate and ascorbate, adipate, alginate, arginate, aspartate, bisulfate, bisulfate, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate, galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate.

And in some specific examples, the compound of Formula I or the pharmaceutically acceptable salt, ester, hydrate or prodrug thereof is administrated to the subject in a form of sol, aromatic water, aerosol, partial inhalant, powder, granule, tablets, ointment, paste, emulsion, suspension, gas dispersion, solid dispersion, microparticle, pill.

And in some specific examples, the compound of Formula I or a pharmaceutically acceptable salt, ester, hydrate or prodrug thereof is administrated at a daily dose of lower than 1000 mg in term of the compound of Formula I.

And in some specific examples, the compound of Formula I or a pharmaceutically acceptable salt, ester, hydrate or prodrug thereof is administrated at a daily dose of 100 mg to 1000 mg in term of the compound of Formula I.

And in some specific examples, the compound of Formula I or a pharmaceutically acceptable salt, ester, hydrate or prodrug thereof is administrated at a daily dose of 100 mg to 500 mg in term of the compound of Formula I.

### EXAMPLES

### Example 1

### Evaluating the Inhibitory Effect of WS-635 on the SARS-CoV-2 Coronavirus at the Cellular Level

WS-635 provided by Waterstone Pharmaceuticals Inc. as powder.

Virus cells are preserved by the Virology Laboratory of the Institute of Microbiology and Epidemiology, Academy of Military Medical Sciences.

SARS-CoV-2 coronavirus isolates (with deposition code: 2019-nCoV BetaCoV/Beijing/AMMS01/2020) are preserved by Institute of Microbiology and Epidemiology, Academy of Military Medical Sciences.

Determination of EC₅₀ of WS-635 by viral nucleic acid quantification:

Human Vero cell was purchased from ATCC (US), The SARS-Cov-2 virus was isolated by Beijing Military Medical Research Institute with the collection deposition code: 2019-nCoV BetaCoV/Beijing/AMMS01/2020.

1). The CC₅₀ of the drug was determined by the method of MTS (Promega). The details are as follows: Vero cells were inoculated into 96 well cell culture plate at 10000/well concentration, and cultured overnight at 37° C. and 5% CO₂. When the cell grew to a full monolayer, 2% FBS DMEM maintenance solution containing different concentrations of the drug (initial concentration of 400 μM) was added to 100 μL/well, and 4 multiple pores were measured at each concentration. Continue to culture and observe the cell status under the microscope every day. Add 20 μL MTS solution until the 4th day of dosing, incubate at 37° C. for 1 hour, and measure the OD490 value.

2). EC₅₀ of the drug was determined by nucleic acid quantitative method. As follows: Vero cells were inoculated into 96 well plates at a concentration of 10000/well one day in advance. Dilute the 2019-nCoV virus with 2% cell maintenance medium and add it to a 96-well plate to make the virus content of each well is 100 TCID₅₀. WS-635 solubilized in DMSO was prepared into 400, 200, 100, 50, 25 and 12.5 μM with DMEM maintenance solution of 2% FBS. The cell culture supernatant was discarded, and T705 (100 μL/well) of different concentrations was added. Each drug had 4 multiple holes, and then 100 TCID₅₀ virus solution was added to each drop. The positive drug control (10 μM Remdesivir (RDV)), virus control and normal cell control groups were set up and cultured in 37° C., 5% CO₂ incubator. On the second day after infection, 50 μL cell supernatant was taken from each pore to extract nucleic acid. The viral load was detected by quantitative RT-PCR and EC₅₀ was calculated by fitting the dose-response curve with graphpad prism 7.

3). Selective Index SI=CC₅₀/EC₅₀.

Test the inhibitory effect of WS-635 on SARS-CoV-2 novel coronavirus at the cellular level by nucleic acid quantification method was used, and calculate the EC₅₀ of the WS-635.

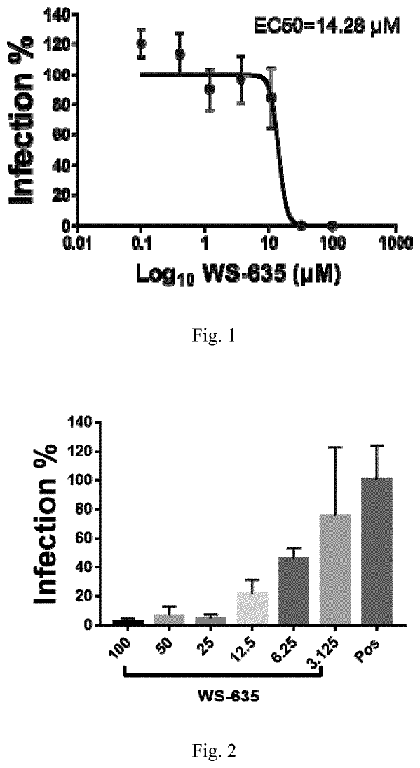

The FIG. 1 shows that WS-635 has an inhibitory effect on the 2019-nCoV new coronavirus at the cellular level, its EC₅₀ is 14.28 μM (Fig). CC₅₀>100 μM, SI>7.

### Example 2

### Ex Vivo Assay of WS-635 for SARS-CoV-2 Replication

Experiment was carried out by Chinese Institute of Virology at a P3 laboratory:

Human Vero Cells (ATCC) were infected with virus SARS-CoV-2 (2019-nCOV BetaCoV/Beijing/AMMS01/2020) to viral concentration of 100 TCID₅₀. Viral RNA load were measured with incubation at different compound concentrations.

Vero cells were inoculated into 96 well plates at a concentration of 10000/well one day in advance. Dilute the 2019-nCoV virus with 2% cell maintenance medium and add it to a 96-well plate to make the virus content of each well is 100 TCID₅₀. WS-635 solubilized in DMSO was prepared into 400, 200, 100, 50, 25 and 12.5 μM with DMEM maintenance solution of 2% FBS. The cell culture supernatant was discarded, and T705 (100 μL/well) of different concentrations was added. Each drug had 4 multiple holes, and then 100 TCID₅₀ virus solution was added to each drop. The positive drug control (10 μM Remdesivir (RDV)), virus control and normal cell control groups were set up and cultured in 37° C., 5% CO₂ incubator. On the second day after infection, 50 μL cell supernatant was taken from each pore to extract nucleic acid. The viral load was detected by quantitative RT-PCR and EC₅₀ was calculated by fitting the dose-response curve with graphpad prism 7.

Inhibition and cytotoxicity curves are shown in FIG. 2 and Table 1. The figure shows that WS-635 inhibits SARS-CoV-2 (2019-nCOV BetaCoV/Beijing/AMMS01/2020) with a dose-dependent manner.

TABLE 1

| CPD ID | EC₅₀ (μM) |
|---|---|
| WS-635 | 5.5 |

### Example 3

### Evaluating the Antiviral Activity of Test Compound WS-635 Against Human Coronavirus (HCoV) 229E in a Cytopathic Effect (CPE) Assay.

### 3.1. Test and Reference Compounds

Test compound WS-635 was provided by the sponsor in dry powders and prepared as 60 mM stock solutions in 100% DMSO solution. Reference compound remdesivir was provided by WuXi AppTec. Compounds were tested at 8 concentrations, 3-fold dilutions, in duplicate for 50% effective concentration (EC₅₀) and 50% cytotoxicity concentration (CC₅₀) determinations. The highest concentrations tested were listed in Table 2. The final concentration of DMSO in cell culture was 0.5%.

TABLE 2
Information of the compounds

| | | | | | The highest testing | The highest testing |
|---|---|---|---|---|---|---|
| Compound | Weight | Purity | | | concentration for | concentration for |
| ID | (mg) | (%) | MW | Aspect | EC₅₀ | CC₅₀ |
| WS-635 | 11.55 | 100 | 1321.8 | Powder | 100 μM | 300 μM |
| Remdesivir | 10.31 | 99 | 602.2 | Powder | 1 μM | 100 μM |

### 3.2. Cell Line and Virus Strain

HCoV 229E (ATCC VR-740) and MRC-5 cells (ATCC CCL-171) were acquired from the ATCC. MRC-5 cells are maintained in the Minimum Essential Medium (Sigma) supplemented with 10% FBS (Hyclone), 1% L-glutamine (Gibco), 1% NEAA (Gibco) and 1% penicillin-streptomycin (Hyclone). Minimum Essential Medium supplemented with 5% FBS, 1% L-glutamine, 1% NEAA and 1% penicillin-streptomycin was used as the assay medium.

### 3.3. Reagents and Instruments

The main reagent used in this assay was luminescent cell viability assay kit CellTiter Glo (Promega). The main instrument used in this assay was Microplate Reader Synergy2 (BioTek).

### 3.4. Methods

The antiviral assay is summarized in Table 3.

TABLE 3
Method of the antiviral assay

| | | Treatment time | Reference | Detection |
|---|---|---|---|---|
| Virus | Cells | (day)/Endpoint | compound | reagent |
| HCoV 229E | MRC-5 | 3/CPE | Remdesivir | CellTiter Glo |

### 3.4.1. HCoV 229E CPE Assay

In 96-well plates, MRC-5 cells were seeded at 20,000 cells per well and cultured at 37° C. and 5% CO₂ overnight. Next day, the medium containing serially diluted compounds and virus (200 TCID₅₀ per well) was added. The resulting cultures were kept at 35° C. and 5% CO₂ for additional 3 days until that virus infection in the virus control (cells infected with virus, without compound treatment) displays significant CPE. Cell viability was measured with CellTiter Glo according to the manufacturer's manual. The luminescent signal was measured by Microplate Reader Synergy2 (Molecular Device). The antiviral activity of each compound was calculated based on the inhibition of CPE at each concentration normalized by the virus control.

3.4.2. Cytotoxicity assay

Cytotoxicity of compounds was assessed under the same conditions but without virus infection, in parallel. Cell viability was measured with CellTiter Glo according to the manufacturer's manual. CC₅₀ values were then calculated based on cytotoxicity at the test concentrations normalized by the medium control (medium only).

### 3.4.3. Data Analysis

Antiviral activity and cytotoxicity of compounds were expressed as % inhibition and % cell viability, respectively, and calculated with the formulas below:

Inhibition (%)=(Raw data _{CPD}−Average _{VC})/(Average _{CC}−Average _{VC})×100

Cell Viability (%)=(Raw data _{CPD}−Average _{MC})/(Average _{CC}−Average _{MC})×100

Where the Raw data _{CPD} indicates the values of the compound-treatment wells; Average _{VC}, Average _{CC} and Average _{MC} indicate the average values of the virus control, cell control (cells without virus infection or compound treatment) and medium control, respectively.

EC₅₀ and CC₅₀ values were calculated using GraphPad Prism software (Version 5) and using the equation log(inhibitor) vs. response with variable slope. SI (CC₅₀/EC₅₀) was then calculated.

### 3.5. Results and Discussions

The antiviral experiment was quality controlled with reference compound. Remdesivir showed expected antiviral activity and effect on cell viability, indicating reliability of the experiment. The antiviral and cytotoxic results of test compounds are summarized in Table 3.

WS-635 showed inhibitory activity against HCoV 229E with an EC₅₀ value of 5.16 μM. It showed obvious cytotoxicity on MRC-5 cells with CC₅₀ and SI value of 46.79 μM and 9.07, respectively.

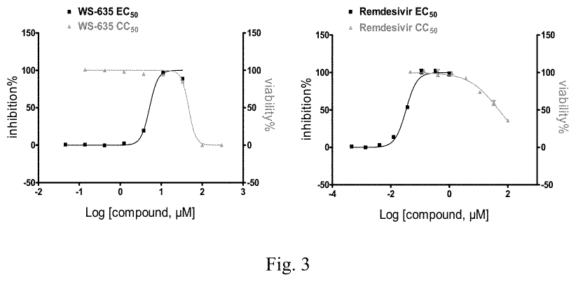

Inhibition and cytotoxicity curves are shown in FIG. 3 and Table 4. The figure shows that WS-635 has obvious cytotoxicity on MRC-5 cells infected with HCoV 229E and works by blocking the spread of the virus.

TABLE 4
EC₅₀ and CC₅₀ values of WS-635

| | | | SI |
|---|---|---|---|
| CPD ID | EC₅₀ (μM) | CC₅₀ (μM) | (CC₅₀/EC₅₀) |
| WS-635 | 5.16 | 46.79 | 9.07 |
| Remdesivir | 0.03 | 49.25 | 1490.62 |

Reference throughout this specification to “an embodiment”, “some embodiments”, “one embodiment”, “another example”, “an example”, “a specific example” or “some examples” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example, “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. A method of treating or preventing Coronaviridae infection in a subject comprising administrating a therapeutically effective amount of a compound of Formula I or a stereoisomer, a tautomer, an N-oxide, a solvate, a metabolite, a pharmaceutically acceptable salt or a prodrug thereof, 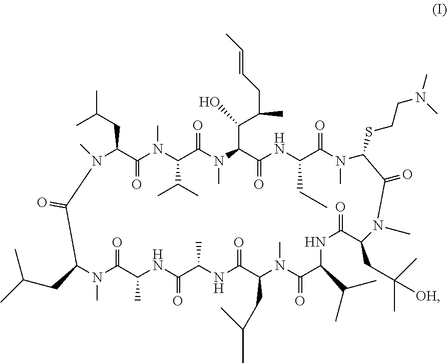 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
and the Coronaviridae comprises at least one selected from 2019-nCov virus, HCov 229E virus, SARS virus, and MERS virus.

2. The method of claim 1, further comprising administering a therapeutically effective amount of at least one other therapeutic agent or composition thereof selected from the group comprising a corticosteroid, an anti-inflammatory signal transduction modulator, a β2-adrenoreceptor agonist bronchodilator, an anticholinergic, a mucolytic agent, hypertonic saline, agent for reducing cytokine storm, other drugs for treating a covid-19 virus infection, or mixtures thereof

3. The method of claim 1, wherein the compound of Formula I or a stereoisomer, a tautomer, an N-oxide, a solvate, a metabolite, a pharmaceutically acceptable salt or a prodrug thereof is administrated to the subject in a form of sol, aromatic water, aerosol, partial inhalant, powder, granule, tablets, ointment, paste, emulsion, suspension, gas dispersion, solid dispersion, micro particle, or pill.

4. The method of claim 1, wherein the pharmaceutically acceptable salt is at least one selected from a group comprising phosphate, hydrochloride, hydrobromide, hydroiodide, sulfate, nitrate, ethanesulfonate, toluenesulfonate and benzenesulfonate, acetate, trifluoroacetate, tartrate, maleate, succinate, citrate, benzoate, salicylate and ascorbate, adipate, alginate, arginate, aspartate, bisulfate, bisulfate, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate, galacturonate, glucoheptaoate, gluconate, glutamate, glycerophosphate, hemisuccinate, hemi sulfate, heptanoate, hexanoate, hippurate, hydrochloride, hydrobromide, hydroiodide, 2-hydroxy ethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate.

5. The method of claim 1, wherein the compound of Formula I or a stereoisomer, a tautomer, an N-oxide, a solvate, a metabolite, a pharmaceutically acceptable salt or a prodrug thereof is administrated to the subject further comprising together with one or more pharmaceutically acceptable carriers, diluents or excipients.

6. The method of claim 1, wherein the compound of Formula I or a pharmaceutically acceptable salt, ester or prodrug thereof is administrated at a daily dose of lower than 1000 mg in term of the compound of Formula I.

7. The method of claim 1, wherein the compound of Formula I or a pharmaceutically acceptable salt, ester or prodrug thereof is administrated at a daily dose of 100 mg to 1000 mg in term of the compound of Formula I.

8. The method of claim 1, wherein the compound of Formula I or a pharmaceutically acceptable salt, ester or prodrug thereof is administrated at a daily dose of 100 mg to 500 mg in term of the compound of Formula I.

9. A pharmaceutical composition to treat or prevent Coronaviridae infection comprising a compound of Formula I or a stereoisomer, a tautomer, an N-oxide, a solvate, a metabolite, a pharmaceutically acceptable salt or a prodrug thereof, 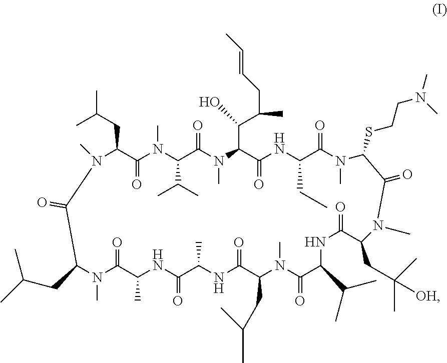 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
and the Coronaviridae comprises at least one selected from 2019-nCov virus, HCov 229E virus, SARS virus, and MERS virus.

10. The pharmaceutical composition of claim 9, further comprising at least one other therapeutic agent or composition thereof selected from the group comprising a corticosteroid, an anti-inflammatory signal transduction modulator, a β2-adrenoreceptor agonist bronchodilator, an anticholinergic, a mucolytic agent, hypertonic saline, agent for reducing cytokine storm, other drugs for treating a covid-19 virus infection, or mixtures thereof.

11. The pharmaceutical composition of claim 9, wherein pharmaceutical composition is in a form of sol, aromatic water, aerosol, partial inhalant, powder, granule, tablets, ointment, paste, emulsion, suspension, gas dispersion, solid dispersion, micro particle, or pill.

12. The pharmaceutical composition of claim 9, further comprising one or more pharmaceutically acceptable carriers, diluents or excipients.

13. The pharmaceutical composition of claim 9, comprising the compound of Formula I or a stereoisomer, a tautomer, an N-oxide, a solvate, a metabolite, a pharmaceutically acceptable salt or a prodrug thereof at a dose of lower than 1000 mg, 800 mg, 600 mg or 500 mg.

14-19. (canceled)
